SEQUENCE LISTING (4 sequences)

SEQ ID NO: 1 - DNA, 27 bp, Artificial Sequence
  a primer for PCR
ttgggactgaattgcactaagatataa

SEQ ID NO: 2 - DNA, 25 bp, Artificial Sequence
  a primer for PCR
gcgttctaaacagttttgcaatttt

SEQ ID NO: 3 - DNA, 18 bp, Artificial Sequence
  a primer for PCR
ctgcaagcatataataca

SEQ ID NO: 4 - protein, 31 aa, Artificial Sequence
  carboxy peptide of long form c-Maf
ITEPTRKLEPSVGYATFWKPQHRVLTSVFTK

(19) United States
(12) Patent Application Publication
(10) Pub. No.: US 2005/0159432 A1
(43) Pub. Date: Jul. 21, 2005

(54) Short form c-Maf transcription factor antagonists for treatment of glaucoma

(75) Inventors: Allan R. Shepard, Fort Worth, TX (US); Nasreen Jacobson, Fort Worth, TX (US); Abbot F. Clark, Arlington, TX (US)

Correspondence Address: HAYNES AND BOONE, LLP, 901 MAIN STREET, SUITE 3100, DALLAS, TX, 75202, US

(73) Assignee: Alcon, Inc., Hunenberg (CH)

(21) Appl. No.: 11/018,283

(22) Filed: Dec. 21, 2004

Related U.S. Application Data
(60) Provisional application No. 60/531,801, filed on Dec. 22, 2003.

(51) Int. Cl. A61K 31/522

(52) U.S. Cl. 514/263.3; 514/263.38

(57) ABSTRACT

The short form version of c-Maf transcription factor is up-regulated in steroid-treated and transforming growth factor beta2-treated trabecular meshwork cells, and is present at elevated levels in glaucomatous versus normal trabecular meshwork cells and in glaucomatous versus normal optic nerve head tissue. Expression of short form c-Maf transcription factor under these conditions indicates a causal or effector role for the factor in primary open-angle and steroid-induced glaucoma pathogenesis. Antagonism of short form c-Maf transcription factor expression and/or activity in the trabecular meshwork or other ocular tissue is provided for inhibiting or alleviating glaucoma pathogenesis. Antagonists include cyclin-dependent kinase 2 inhibitors.

This application claims the benefit of U.S. Provisional Patent Application No. 60/531,801, filed Dec. 22, 2003, which is incorporated by reference in its entirety.

### FIELD OF THE INVENTION

The present invention relates to the field of prophylactic agents and therapeutics for glaucoma, particularly for primary open angle glaucoma and steroid-induced glaucoma.

### BACKGROUND OF THE INVENTION

The trabecular meshwork (TM) is a complex tissue including endothelial cells, connective tissue, and extracellular matrix located at the angle between the cornea and iris that provides the normal resistance required to maintain an intraocular pressure (IOP). An adequate intraocular pressure is needed to maintain the shape of the eye and to provide a pressure gradient to allow for the flow of aqueous humor to the avascular cornea and lens. Excessive IOP, commonly present in glaucoma, has deleterious effects on the optic nerve, leads to loss of retinal ganglion cells and axons, and results in progressive visual loss and blindness if not treated. Glaucoma is one of the leading causes of blindness worldwide.

Primary glaucomas result from disturbances in the flow of intraocular fluid that has an anatomical or physiological basis. Secondary glaucomas occur as a result of injury or trauma to the eye or a preexisting disease. Primary open angle glaucoma (POAG), also known as chronic or simple glaucoma, represents ninety percent of all primary glaucomas. POAG is characterized by the degeneration of the trabecular meshwork, resulting in abnormally high resistance to fluid drainage from the eye. A consequence of such resistance is an increase in the IOP that is required to drive the fluid normally produced by the eye across the increased resistance.

Certain drugs such as prednisone, dexamethasone, and hydrocortisone are known to induce glaucoma by increasing IOP. Further, the mode of administration appears to affect IOP. For example, ophthalmic administration of dexamethasone leads to greater increases in IOP than does systemic administration. Glaucoma that results from the administration of steroids is termed steroid-induced glaucoma.

Current anti-glaucoma therapies include lowering IOP by the use of suppressants of aqueous humor formation or agents that enhance uveoscleral outflow, laser trabeculoplasty, or trabeculectomy which is a filtration surgery to improve drainage. Pharmaceutical anti-glaucoma approaches have exhibited various undesirable side effects. For example, miotics such as pilocarpine can cause blurring of vision and other negative visual side effects. Systemically administered carbonic anhydrase inhibitors can also cause nausea, dyspepsia, fatigue, and metabolic acidosis. Further, certain beta-blockers have increasingly become associated with serious pulmonary side effects attributable to their effects on beta-2 receptors in pulmonary tissue. Sympathomimetics cause tachycardia, arrhythmia and hypertension. Such negative side effects may lead to decreased patient compliance or to termination of therapy.

More importantly, the current anti-glaucoma therapies do not directly address the pathological damage to the trabecular meshwork, the optic nerve, and loss of retinal ganglion cells and axons, which continues unabated. In view of the importance of glaucoma, and the inadequacies of prior methods of treatment, it would be desirable to have an improved method of treating glaucoma that would address the underlying causes of its progression.

### SUMMARY OF THE INVENTION

The present invention relates to a method of treatment for primary open angle glaucoma or steroid-induced glaucoma in a subject at risk for developing primary open angle glaucoma or steroid-induced glaucoma or having symptoms thereof. The method comprises administering to the subject an effective amount of a composition comprising an antagonist of short-form c-Maf transcription factor and an acceptable carrier.

According to the present invention, the short form version of c-Maf transcription factor has been identified as up-regulated in steroid-treated and transforming growth factor-β2 (TGFβ2)-treated trabecular meshwork (TM) cells, as present at elevated levels in glaucomatous versus normal optic nerve head tissue, and as present at elevated levels in glaucomatous versus normal TM cells. Expression of short form c-Maf transcription factor under these conditions indicates a causal or effector role for the factor in primary open-angle and steroid-induced glaucoma pathogenesis. The methods of the present invention involve antagonism of short form c-Maf transcription factor transcription, expression and/or activity in the trabecular meshwork or other ocular tissue, such as optic nerve head tissue, so as to inhibit or alleviate glaucoma pathogenesis.

The antagonist of the present invention interferes with short-form c-Maf transcription factor transcription or expression. In one embodiment, the antagonist of short-form c-Maf transcription factor comprises a purine analog having inhibitory activity for cdk2 cyclin-dependent kinase. The antagonist may comprise purvalanol A, purvalanol B, amino-purvalanol, olomoucine, N9-isopropylolomoucine, roscovitine, methoxy-roscovitine, combinations thereof, or salts thereof, for example.

According to another embodiment, the antagonist having inhibitory activity for cdk2 cyclin-dependent kinase is non-purine based and is an indirubin, oxindole, indenopyrazole, pyridopyrimidine, anilinoquinazoline, aminothiazole, flavopiridol, staurosporine, paullone, hymenialdisine, combinations thereof and salts thereof, for example.

The use of antagonists of the expression or activity of the short form of c-Maf as therapeutic agents to protect or rescue patients from damage caused by the glaucoma disease process addresses the progression of the disease in addition to symptoms of the disease, i.e., the pathogenic process is altered as a result of treatment. The short form c-Maf expression or activity antagonists are useful for the treatment of POAG and steroid-induced glaucoma. The identification of short-form c-Maf transcription factor as a player in glaucoma pathogenesis and the use of expression or activity inhibitors as presented herein has not previously been described.

### BRIEF DESCRIPTION OF THE DRAWINGS

FIG. 1. QPCR analysis of short-form c-Maf expression in SGTM2697 pooled cells demonstrates TGFβ2-induced gene expression upregulated 16-fold as compared to the control.

FIG. 2. QPCR analysis of short-form c-Maf expression in TM70A cells demonstrates dexamethasone-induced gene expression upregulated 2.1-fold on day one and 3.2-fold on day 14 as compared to the control.

FIG. 3. QPCR analysis of short-form c-Maf expression in SGTM2697 (P6) cells in the presence and absence of purvalanolA for basal and TGFβ2-induced cells.

### DETAILED DESCRIPTION OF THE INVENTION

The present invention relates to the use of agents to antagonize short form c-Maf transcription factor expression and/or activity for the treatment of glaucoma. Human genome microarrays were hybridized to normal and glaucomatous RNA and the short form c-Maf transcription factor gene was upregulated in the glaucoma cells as compared to the normal cells.

Maf-related genes have been identified as important players in lens and anterior segment development (Yoshida, et al. (1997), Invest Opthalmol Vis Sci 38(12): 2679-83; Ogino et al. (1998), Science 280(5360): 115-8; Kawauchi, et al. (1999), J Biol Chem 274(27): 19254-60; Kim, et al. (1999), Proc Natl Acad Sci USA 96(7): 3781-5; Ring, et al. (2000), Development 127(2): 307-17; Ishibashi et al. (2001), Mech Dev 101(1-2): 155-66; Jamieson, et al. (2002), Hum Mol Genet 11(1): 33-42; Reza, et al. (2002), Mech Dev 116(1-2): 61-73). c-Maf has been shown to activate crystallin gene expression, is activated by the glaucoma gene product Pax6 (Sakai, et al. (2001), Nucleic Acids Res 29(5): 1228-37; Yoshida, et al. (2001) Curr Eye Res 23(2): 116-9), and is positively autoregulated by its own gene product. Mice lacking c-Maf are microphthalmic with defective lens formation whereas heterozygous null mutants undergo relatively normal ocular development (Kim, e al. (1999), Proc Natl Acad Sci USA 96(7): 3781-5).

c-Maf is a basic region leucine zipper (bZIP) transcription factor. Maf family members have ≦40% homology in the basic domain of their bZIP motifs. Short, single-exon (373 amino acids) and long, two-exon (403 amino acids) forms of c-Maf exist, but their functional distinction remains unknown. The short form of c-Maf ends with a methionine at the C-terminus. The additional carboxy terminal amino acid sequence for the long form is ITEPTRKLEPSVGYATFWKPQHRVLTSVFTK, SEQ ID NO: 4. As used herein, the term “short-form c-Maf transcription factor” means the gene that encodes short-form c-Maf transcription factor or the protein product of 373 amino acids of the protein sequence deposited under Gen Bank accession no. AF055376.

U.S. Pat. No. 6,274,338, to Glimcher et al., the entire disclosure of which is incorporated herein by reference, discloses nucleic acid sequence and protein sequence information for human c-Maf, as well as antisense molecules and anti-cMaf antibodies. The sequence of the cMaf of U.S. Pat. No. 6,274,338 is located in GenPept as accession # AAE79064. This sequence matches the long-form c-Maf with the exception of several amino acid mismatches, including a 3 amino acid deletion at amino acids 241-243 when compared with the protein sequence contained in GenBank numbers AF055376 (short form sequence) and AF055377 (long form sequence).

Antagonists of Short Form c-Maf Transcription Factor:

Antagonists of short form c-Maf transcription factor include agents that decrease transcription of the short form gene, inhibit short form expression, or inhibit short form activity, for example. In particular, it has been found that cdk2 cyclin-dependent kinase inhibitors, particularly purine analogs, downregulate transcription of the short form c-Maf transcription factor. Table 1 provides a listing of antagonists of short form c-Maf transcription factor having inhibitory activity for cdk2.

TABLE 1
Antagonists of Short Form c-Maf Transcription Factor

| Antagonist | Reference for cdk2 inhibitory activity |
|---|---|
| Purine Analogs | |
| Purvalanols such as 2-(1R-Isopropyl-2- | Gray, N. S. et al., Science, 281, 533-538 |
| hydroxyethylamino)-6-(3-chloroanilino)-9- | (1998); |
| isopropylpurine having a molecular formula | Chang, Y. T. et al., Chem. Biol., 6, 361-375 |
| C₁₉H₂₅ClN₆O available from Sigma-Aldrich under | (1999). |
| the trade name Purvalanol A (#P4484, Sigma- | |
| Aldrich, St. Louis, MO), | |
| Purvalanol B, aminopurvalanol, compound 52 | |
| (where isopropyl of purvalanol A is replaced with | |
| H) | |
| 2-(Hydroxyethylamino)-6-benzylamino-9- | Vesely, J., et al., (1994) Eur. J. Biochem., 224, |
| methylpurine having a molecular formula | 771-86, 11; |
| C₁₅H₁₈N₆O available from Sigma-Aldrich under | Brooks, E. E., et al., (1997) J. Biol. Chem., 272, |
| the trade name Olomoucine (#O0886), | 29207-11 |
| 2-(2'-Hydroxyethylamino)-6-benzylamino-9- | |
| isopropylpurine having a molecular formula | |
| C₁₇H₂₂N₆O available from Sigma-Aldrich under | |
| the trade name N⁹-isopropylolomoucine (#I0763); | |
| CVT-313 | |
| 6-(Benzylamino)-2(R)-[[1- | Wang, D. et al., J. Virol., 75, 7266-7279 |
| (hydroxymethyl)propyl]amino]-9-isopropylpurine | (2001); McClue, S. J. et al., Int. J. Cancer, 102, |
| 2-(R)-[[9-(1-methylethyl)-6- | 463-468 (2002); |
| [(phenylmethyl)amino]-9H-purin-2-yl]amino]-1- | Meijer, L., et al., (1997) Eur. J. Biochem., 243, |
| butanol having a molecular formula of C₁₉H₂₆N₆O | 527-36 |
| available from Sigma-Aldrich under the trade | |
| name Roscovitine (#R7772), | |
| methoxyroscovitine | |
| Purine analog N2-(cis-2-Aminocyclohexyl)-N6- | Imbach, P. et al., Bioorg. Med. Chem. Lett., 9, |
| (3-chlorophenyl)-9-ethyl-9H-purine-2,6-diamine | 91-96 (1999); |
| having a molecular formula of C₁₉H₂₄ClN₇ | Dreyer, M. K. et al., J. Med. Chem., 44, 524-530 |
| available from Sigma-Aldrich under the trade | (2001). |
| name CGP74514 (#C3353) | |
| CGP79807, a purine analog of CGP74514 (supra) | Imbach, P. et al., Bioorg. Med. Chem. Lett., 9, |
| where Cl is replaced with CN, OH is removed, | 91-96 (1999); |
| and the ortho position of cyclohexane ring is NH₂ | Dreyer, M. K. et al., J. Med. Chem., 44, 524-530 |
| | (2001). |
| purine analog such as O6-cyclohexylmethyl | Arris, C. E. et al., J. Med. Chem., 43, 2797-2804 |
| guanine NU2058 | (2000); |
| | Davies et al, Nature Structural Biology, 9:10, |
| | 745-749, 2002 |
| purine analog such as NU6102 | Arris, C. E. et al., J. Med. Chem., 43, 2797-2804 |
| | (2000); Davies, T. G. et al., Nat. Struct. |
| | Biol., 9, 745-749 (2002). |
| isopentenyl-adenine | Vesely, J., et al., (1994) Eur. J. Biochem., 224, |
| | 771-86 |
| Nonpurine based agents | |
| Indirubins such as indirubin-3'-monoxime having | Davies, T. G. et at., Structure, 9, 389-397 |
| a molecular formula of C₁₆H₁₁N₃O₂ available from | (2001); |
| Sigma-Aldrich under the trade name (#I0404), | Marko, D. et al., Br. J. Cancer, 84, 283-289 |
| indirubin 5-sulfonate, 5-chloro indirubin | (2001); |
| | Hoessel, R., et al., (1999) Nat. Cell Biol., 1, |
| | 60-7; |
| | PCT/US02/30059 to Hellberg et al., published |
| | as WO 03/027275. |
| Oxindole 1 of Fischer as referenced in column 2 | Porcs-Makkay, M., et al., Tetrahedron 2000, |
| of this table, (#IN118, JMAR Chemical, | 56, 5893; Org. Process Res. Dev. 2000, 4, 10 |
| Indenopyrazoles | Nugiel, D. A. et al., J. Med. Chem., 44, 1334-1336 |
| | (2001); Nugiel, D. A. et al., J. Med. |
| | Chem., 45, 5224-5232 (2002); Yue, E. W. et |
| | al., J. Med. Chem., 45, 5233-5248 (2002). |
| Pyrido(2,3-d)pyrimidine-7-ones, compound 3 of | Barvian, M. et al., J. Med. Chem., 43, 4606-4616 |
| Fischer | (2000); Toogood, P. L., Med. Res. Rev., |
| | 21, 487-498 (2001). |
| Quinazolines such as anilinoquinazoline | Sielecki, T. M. et al., Bioorg. Med. Chem. |
| | Lett., 11, 1157-1160 (2001); |
| | Mettey et al., J. Med. Chem. 2003, 46, 222-236. |
| Thiazoles such as fused thiazole, 4-{[(7-Oxo-6,7- | Davis, S. T. et al., Science, 291, 134-137 |
| dihydro-8H-[1,3]thiazolo[5,4-e]indol-8- | (2001); |
| ylidene)methyl]amino}-N-(2- | PCT/US02/30059 to Hellberg et al., published |
| pyridyl)benzenesulfonamide having a molecular | as WO 03/027275. |
| formula of C₂₁H₁₅N₅O₃S₂ available from Sigma- | |
| Aldrich under the trade name GW8510 (#G7791) | |
| Flavopiridols such as flavopiridol (L86 8275; | Carlson, B. A., et al., (1996) Cancer Res., 56, |
| NCS 649890, National Cancer Institute, Bethesda, | 2973-8 |
| MD) and a dechloro derivative | |
| Alkaloids such as Staurosporine (#S1016, A. G. | Rialet, V., et al., (1991) Anticancer Res., 11, |
| Scientific, San Diego, CA) or UCN-01 (7- | 1581-90; |
| hydroxystaurosporine) National Cancer Institute, | Wang, Q., et al., (1995) Cell Growth Differ., 6, |
| Bethesda, MD | 927-36, Akiyama, T., et al., (1997) Cancer |
| | Res., 57, 1495-501, Kawakami, K., et al., |
| | (1996) Biochem. Biophys. Res. Commun., 219, |
| | 778-83 |
| Paullones such as 9-Bromo-7,12-dihydro- | Zaharevitz, D. W. et al., Cancer Res., 59, 2566-2569 |
| indolo[3,2-d][1]benzazepin-6(5H)-one having a | (1999); Schultz, C. et al., J. Med. Chem., |
| molecular formula of C₁₆H₁₁BrN₂O available from | 42, 2909-2919 (1999); |
| Sigma-Aldrich under the trade name kenpaullone | Zaharevitz, D. W., et al., (1999) Cancer Res., |
| (#K3888), or 9-Nitro-7,12-dihydroindolo-[3,2- | 59, 2566-9; |
| d][1]benzazepin-6(5)-one having a molecular | PCT/US02/30059 to Hellberg et al., published |
| formula of C₁₆H₁₁N₃O₃ available from Sigma- | as WO 03/027275. |
| Aldrich under the trade name alsterpaullone | |
| (#A4847) | |
| CGP 41251, an alkaloid | Begemann, M., et al., (1998) Anticancer Res., |
| | 18, 2275-82; |
| | Fabbro et al., Pharmacol Ther. 1999 May-Jun; |
| | 82(2-3): 293-301 |
| Hymenialdisines such as 10z-hymenialdisine | Meijer, L., et al., (1999) Chemistry & Biology, |
| having a molecular formula of C₁₁H₁₀BrN₅O₂ | 7, 51-63; |
| available from Biochemicals.net, a division of | PCT/US02/30059 to Hellberg et al., published |
| A.G. Scientific, Inc. (San Diego, CA) (H-1150) | as WO 03/027275. |
| CGP60474, a phenylaminopyrimidine | 21; WO95/09853, Zimmermann et al., |
| | September 21, 1994 |
| Thiazolopyrimidine 2 | Attaby et al., Z. Naturforsch. 54b, 788-798 |
| | (1999) |
| Diarylurea | Honma, T. et al., J. Med. Chem., 44, 4628-4640 |
| | (2001), Honma, T. et al., J. Med. Chem., |
| | 44, 4615-4627 (2001). |
| (2R)-2,5-Dihydro-4-hydroxy-2-[(4-hydroxy-3-(3- | Kitagawa, M. et al., Oncogene, 8, 2425-2432 |
| methyl-2-butenyl)phenyl)methyl]-3-(4- | (1993). |
| hydroxyphenyl)-5-oxo-2-furancarboxylic acid | |
| methyl ester having a molecular formula of | |
| C₂₄H₂₄O₇ available from Sigma-Aldrich under the | |
| trade name Butyrolactone-I (B7930) | |
| Aloisine A, Cat. No. 128125 (Calbiochem, San | Mettey et al., J. Med. Chem. 2003, 46, 222-236 |
| Diego, CA) | |

Further cdk2 inhibitory agents are described in U.S. Pat. No. 6,573,044, to Gray et al., Rosania et al., Exp. Opin. Ther. Patents (2000) 10(2); 215-230, in particular, section 3 to small molecule inhibitors, Fischer, P. M., Celltransmissions 19:1, pg 3-9, March, 2003. One of skill in the art in light of the present specification will appreciate that agents can be a racemic mixture, or either diastereomer or enantiomer, according to substituents.

Despite their chemical variety, many of the compounds of Table 1 compete with ATP for the binding site in the cyclin/cdk2 complex. For example, results of structural analysis have shown that the purine portion of many of the purine inhibitors binds to the adenine-binding pocket of the cdk2, preventing binding of its true ligand. A planar heterocyclic ring system appears to be a structural feature common to many cdk2 inhibitors.

An assay for an antagonist of short-form c-Maf transcription factor comprises combining a candidate antagonist with the c-Maf transcription factor gene in a background that allows transcription and expression to occur. An amount of c-Maf transcription factor present or activity less than that in the absence of the candidate antagonist indicates that the candidate antagonist is, in fact, an antagonist of c-Maf.

Mode of Administration:

The antagonist may be delivered directly to the eye (for example: topical ocular drops or ointments; slow release devices in the cul-de-sac or implanted adjacent to the sclera or within the eye; periocular, conjunctival, sub-Tenons, intracameral, intravitreal, or intracanalicular injections) or systemically (for example: orally; intravenous, subcutaneous or intramuscular injections; parenterally, dermal delivery) using techniques well known by those skilled in the art. It is further contemplated that the antagonists of the invention may be formulated in intraocular insert or implant devices. Intracameral injection may be through the cornea into the anterior chamber to allow the agent to reach the trabecular meshwork. Intracanalicular injection may be into the venous collector channels draining Schlemm's canal or into Schlemm's canal.

Subject:

A subject treated for primary open angle glaucoma or for steroid-induced glaucoma as described herein may be a human or another animal at risk of developing primary open angle glaucoma or steroid-induced glaucoma or having symptoms of primary open angle glaucoma or steroid-induced glaucoma.

Formulations and Dosage:

The antagonists of the present invention can be administered as solutions, suspensions, or emulsions (dispersions) in a suitable ophthalmic carrier. The following are examples of possible formulations embodied by this invention.

| | Amount in weight % |
|---|---|
| c-Maf transcription factor inhibitor | 0.01-5; 0.01-2.0; 0.5-2.0 |
| Hydroxypropylmethylcellulose | 0.5 |
| Sodium chloride | .8 |
| Benzalkonium Chloride | 0.01% |
| EDTA | 0.01 |
| NaOH/HCl | qs pH 7.4 |
| Purified water | qs 100 mL |
| c-Maf transcription antagonist | 0.00005-0.5; 0.0003-0.3; |
| | 0.0005-0.03; 0.001 |
| Phosphate Buffered Saline | 1.0 |
| Benzalkonium Chloride | 0.01 |
| Polysorbate 80 | 0.5 |
| Purified water | q.s. to 100% |
| c-Maf transcription antagonist | 0.001 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCL and/or NaOH | pH 7.3-7.4 |
| Purified water | q.s. to 100% |
| c-Maf transcription antagonist | 0.0005 |
| Phosphate Buffered Saline | 1.0 |
| Hydroxypropyl-β-cyclodextrin | 4.0 |
| Purified water | q.s. to 100% |

In a further embodiment, the ophthalmic compositions are formulated to provide for an intraocular concentration of about 0.1-100 nanomolar (nM) or, in a further embodiment, 1-10 nM of the antagonist. Topical compositions are delivered to the surface of the eye one to four times per day according to the routine discretion of a skilled clinician. The pH of the formulation should be 4-9, or 4.5 to 7.4. Systemic formulations may contain about 10 to 1000 mg of the antagonist.

An “effective amount” refers to that amount of c-Maf antagonist that is able to disrupt short form c-Maf expression or activity. Such disruption leads to lowered intraocular pressure, and lessening of symptoms of glaucoma in a subject exhibiting symptoms of primary open angle glaucoma or steroid-induced glaucoma. Such disruption delays or prevents the onset of symptoms in a subject at risk for developing glaucoma. The effective amount of a formulation may depend on factors such as the age, race, and sex of the subject, or the severity of the glaucoma, for example. In one embodiment, the antagonist is delivered topically to the eye and reaches the trabecular meshwork, retina or optic nerve head at a therapeutic dose thereby ameliorating the glaucoma disease process.

While the precise regimen is left to the discretion of the clinician, the resulting solution or solutions are preferably administered by placing one drop of each solution(s) in each eye one to four times a day, or as directed by the clinician.

Acceptable Carriers:

An ophthalmically acceptable carrier refers to those carriers that cause at most, little to no ocular irritation, provide suitable preservation if needed, and deliver one or more c-Maf antagonists of the present invention in a homogenous dosage. For ophthalmic delivery, a c-Maf transcription inhibitor may be combined with ophthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the inhibitor in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like, may be added to the compositions of the present invention to improve the retention of the compound.

In order to prepare a sterile ophthalmic ointment formulation, the c-Maf antagonist is combined with a preservative in an appropriate vehicle, such as mineral oil, liquid lanolin, or white petrolatum. Sterile ophthalmic gel formulations may be prepared by suspending the c-Maf antagonist in a hydrophilic base prepared from the combination of, for example, CARBOPOL®-940 (BF Goodrich, Charlotte, N.C.), or the like, according to methods known in the art for other ophthalmic formulations. VISCOAT® (Alcon Laboratories, Inc., Fort Worth, Tex.) may be used for intraocular injection, for example. Other compositions of the present invention may contain penetration enhancing agents such as cremephor and TWEEN® 80 (polyoxyethylene sorbitan monolaureate, Sigma Aldrich, St. Louis, Mo.), in the event the c-Maf antagonists are less penetrating in the eye.

### EXAMPLE 1

### RNA Isolation from Human Trabecular Meshwork Tissue and Cells

Human trabecular meshwork (TM) cells were derived from donor eyes (Central Florida Lions Eye and Tissue Bank, Tampa, Fla.) and cultured as previously described (Steely, et al. (1992), Invest Ophthalmol Vis Sci 33(7): 2242-50; Wilson, et al. (1993), Curr Eye Res 12(9): 783-93; Clark, et al. (1994), Invest Ophthalmol Vis Sci 35(1): 281-94.; Dickerson, et al. (1998), Exp Eye Res 66(6): 731-8; Wang, et al. (2001), Mol Vis 7: 89-94). TM cells were derived from pools of four each of either normal or glaucoma cell lines. Total RNA was isolated from TM cells from each pool using TRIZOL® reagent according to the manufacturer's instructions (Invitrogen, Carlsbad, Calif.).

### EXAMPLE 2

### Affymetrix GeneChip Analysis

Reverse transcription, second-strand cDNA synthesis and biotin-labeling of amplified RNA were carried out according to standard Affymetrix protocols. Human genome U133A and U133B GENECHIPS® (Affymetrix, Santa Clara, Calif.) were hybridized, washed and scanned according to standard Affymetrix protocols. Hybridized GENECHIP® arrays were scanned with a GENEARRAY® scanner (Agilent Technologies, Palo Alto, Calif.). Raw data were collected and analyzed using Affymetrix Microarray Suite software.

Filtering of microarray data was done using GENESPRING® software (Silicon Genetics, Redwood City, Calif.). For each experiment, data were normalized per chip by dividing each measurement by the 50th percentile of all signal intensity measurements for that chip. The expression ratio for each gene was calculated by dividing the normalized signal per gene in the treated or diseased sample by the median for that gene in the control sample for each experiment. Genes were selected for an expression level above the statistical background by using the Cross-Gene Error Model and setting the baseline equal to the unique base/proportional value for each experiment. Only genes that were flagged as present/marginal on the Affymetrix U133A GENECHIP® in all experimental conditions were considered for analysis. The c-Maf short form gene is represented only once on the U133A GENECHIP® as probe set 209348_s_at. Short-form c-Maf was expressed at least two-fold higher in disease or treated vs. control conditions.

### EXAMPLE 3

### Quantitative PCR

First strand cDNA was generated from 1 μg of total RNA using random hexamers and TAQMAN® Reverse Transcription reagents according to the manufacturer's instructions (Applied Biosystems, Foster City, Calif.). The 100 μl reaction was subsequently diluted 20-fold to achieve an effective cDNA concentration of 0.5 ng/μl.

Measurement of short form c-Maf gene expression was performed by quantitative real-time RT-PCR (QPCR) using an ABI PRISM® 7700 Sequence Detection System (Applied Biosystems) essentially as described Shepard, et al. (2001) Invest Ophthalmol Vis Sci 42(13): 3173-81. Primers for short form-specific c-Maf amplification (Genbank accession #AF055376) were designed using PRIMER EXPRESS® software (Applied Biosystems). Forward and reverse primer sequences were TTGGGACTGAATTGCACTAAGATATAA, SEQ ID NO:1, (nucleotides 3773-3799) and GCGTTCTAAACAGTTTTGCAATTTT, SEQ ID NO:2, (nucleotides 3823-3847), and the minor groove binding probe sequence was CTGCAAGCATATAATACA, SEQ ID NO:3, (nucleotides 3801-3818). 6FAM was bound to the 5′ end of the minor groove binding probe and refers to the type of fluorophore attached to the TAQMAN® probe. Other choices for the fluorophore are the JOE™ Fluorophore (Applied Biosystems) or VIC™ fluorophore (Applied Biosystems). The “Minor Groove Binding Non-Fluorescent Quencher” was bound to the 3′ end of the probe and is used to quench the fluorescence from 6FAM. Amplification of the 75-bp c-Maf amplicon was normalized to 18S rRNA levels using 1× pre-developed 18S rRNA primer/probe set (20× 18S MASTER MIX®; Applied Biosystems). c-Maf QPCR consisted of 1× TAQMAN® Universal Mix (Applied Biosystems), 900 nM primer and 100 nM probe concentrations, and 2.5 ng cDNA in a final volume of 50 μl. Thermal cycling conditions consisted of 50° C., 2 min, 95° C. 10 min followed by 40 cycles at 95° C., 15 sec, 60° C., 1 min. Quantification of relative cDNA concentrations was done using the relative standard curve method as described in PE Biosystems User Bulletin #2, ABI PRISM® 7700 Sequence Detection System, 2001 (Applied BioSystems). Data analysis was performed with SDS software version 1.9.1 (Applied Biosystems) and MS Excel 97 (Microsoft). Human reference total RNA (Stratagene, La Jolla, Calif.) was used for generating the standard curve. QPCR data are presented as mean ±SEM of the c-Maf/18S normalized ratio.

### EXAMPLE 4

### TGFβ2-Induced c-Maf Gene Expression in Trabecular Meshwork Cells

The present example demonstrates that the short form of c-Maf was differentially upregulated in transforming growth factor beta 2-induced glaucomatous cells using quantitative PCR analysis.

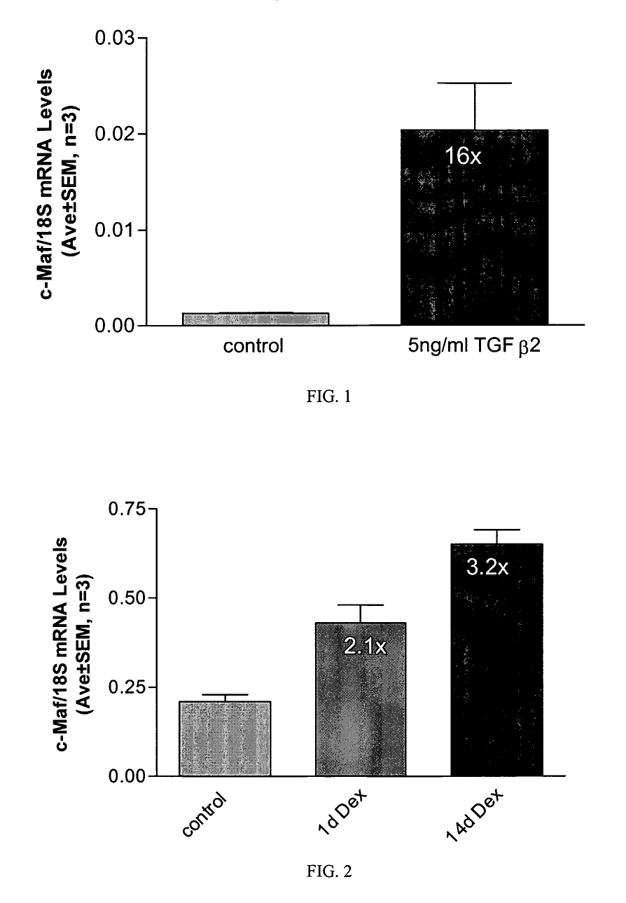

Short form c-Maf gene expression was analyzed using the Affymetrix U133A GENECHIP® analysis described in Example 2 of a pool of glaucomatous trabecular meshwork cells designated SGTM2697. The glaucomatous cells were treated for 16 hours with 5 ng/ml transforming growth factor beta 2 (TGFβ2) for induction of gene expression. Gene expression of the short form of c-Maf was identified as upregulated. Verification of c-Maf upregulation was performed by QPCR as described in Example 3 using CDNA derived from the pooled ±TGFβ2-treated SGTM2697 cell RNA used for the Affymetrix GENECHIP® analysis. Short form c-Maf was upregulated 16-fold by TGFβ2 compared to control as shown in FIG. 1. Data of FIG. 1 are presented as the normalized ratio of c-Maf to ribosomal 18S mRNA levels (Mean ±SEM, n=3).

### EXAMPLE 5

### Dexamethasone-Induced c-Maf Gene Expression in Trabecular Meshwork Cells

The present example demonstrates that the short form of c-Maf was differentially upregulated in dexamethasone-induced glaumomatous cells using quantitative PCR analysis.

Short form c-Maf gene expression was analyzed using the Affymetrix U133A GENECHIP® analysis described in Example 2 for trabecular meshwork cells designated TM70A. The cells were treated 1 day or 14 days with 10⁻⁷ M dexamethasone (Dex). Gene expression of the short form of c-Maf was identified as upregulated. Verification of c-Maf upregulation was performed by QPCR as described in Example 3 using cDNA derived from the ±Dex-treated TM70A cell RNA used for the Affymetrix GENECHIP® analysis. Short form c-Maf was upregulated 2.1-fold at day one and 3.2-fold by day 14 of Dex treatment compared to control as shown in FIG. 2. Data are presented as the normalized ratio of c-Maf to ribosomal 18S mRNA levels (Mean ±SEM, n=3).

### EXAMPLE 6

### Small Molecule Inhibition of Basal and TGFβ2-Induced Short-Form c-Maf Gene Expression in Trabecular Meshwork Cells

The present example demonstrates that a cdk2 inhibitor is an antagonist of short-form c-Maf gene expression.

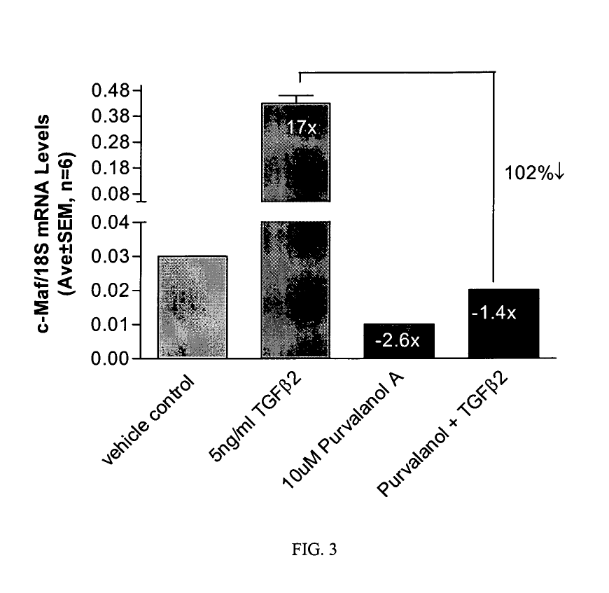

The effect of small molecule inhibition on short-form c-Maf gene expression was analyzed by QPCR analysis as described in Example 2 in glaucomatous trabecular meshwork (Passage 6) cells designated SGTM2697. The cells were treated with or without 5ng/ml TGFβ2 and the cdk2/cyclin A inhibitor purvalanol A for 16 hours (Hardcastle, et al. (2002) Annu Rev Pharmacol Toxicol 42:325-348). Basal c-Maf levels were downregulated 2.6-fold by purvalanol A treatment as shown in FIG. 3. TGFβ2-treated c-Maf (upregulated 17-fold) was completely abolished by purvalanol A co-treatment as shown in FIG. 3. Data of FIG. 3 are presented as the normalized ratio of c-Maf to ribosomal 18S mRNA levels (Mean ±SEM, n=6). The y-axis of FIG. 3 has a lower scale from 0.00 to 0.03 and an upper scale from 0.08 to 0.48.

As supported by the purvalanol A inhibiton of short-form c-Maf gene expression set forth above, the present invention provides further cyclin-dependent kinase 2 inhibitors as described herein for use as antagonists of expression of the short form of c-Maf. Such antagonists are useful as prophylactic or therapeutic agents to protect from or treat damage caused by the glaucoma disease process.

### EXAMPLE 7

### Short Form c-Maf Transcription Factor in Glaucomatous Optic Nerve Head Tissue

The short form version of c-Maf transcription factor is present at elevated levels in glaucomatous versus normal optic nerve head tissue using the Affymetrix GENECHIP® microarray analysis. Optic nerve head tissue was derived from pools of either four normal or five glaucomatous donor eyes. Total RNA was isolated from optic nerve head tissue using TRIZOL® reagent according to the manufacture's instructions (Invitrogen). Expression of short form c-Maf in these conditions further indicates a causal or effector role on the part of the factor in glaucoma pathogenesis. Antagonism of short form c-Maf transcription factor expression and/or activity within ocular tissue is provided for inhibiting or alleviating glaucoma pathogenesis and for providing neuroprotection for the retina and optic nerve.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more”.

1. A method of treatment for primary open angle glaucoma or steroid-induced glaucoma in a subject, the method comprising administering to the subject an effective amount of a composition comprising an antagonist of short-form c-Maf transcription factor and an acceptable carrier.

2. The method of claim 1 wherein the treatment is for primary open angle glaucoma.

3. The method of claim 1. wherein the treatment is for steroid-induced glaucoma.

4. The method of claim 1 wherein the subject is at risk for developing primary open angle glaucoma or steroid-induced glaucoma.

5. The method of claim 1 wherein the subject has symptoms of primary open angle glaucoma or steroid-induced glaucoma.

6. The method of claim 1, wherein the antagonist of short-form c-Maf transcription factor interferes with transcription of the c-Maf gene.

7. The method of claim 1 wherein the antagonist of short-form c-Maf transcription factor comprises a purine analog having inhibitory activity for cdk2 cyclin-dependent kinase.

8. The method of claim 7 wherein the antagonist comprises purvalanol A, purvalanol B, amino-purvalanol, olomoucine, N9-isopropylolomoucine, roscovitine, methoxy-roscovitine, combinations thereof, or salts thereof.

9. The method of claim 7 wherein the antagonist comprises purvalanol A, purvalanol B, combinations thereof, or salts thereof.

10. The method of claim 7 wherein the antagonist comprises purvalanol A.

11. The method of claim 1 wherein the antagonist of short-form c-Maf transcription factor has inhibitory activity for cdk2 cyclin-dependent kinase and is selected from the group consisting of indirubins, oxindoles, indenopyrazoles, pyridopyrimidines, anilinoquinazolines, aminothiazoles, flavopiridols, staurosporines, paullones, hymenialdisines, combinations thereof and salts thereof.

12. The method of claim 1, wherein the administering is by intraocular injection, implantation of a slow release delivery device, or topical, oral, or intranasal administration.

13. The method of claim 1, wherein the administering is by topical administration.

14. A method of treatment for primary open angle glaucoma in a subject, the method comprising administering to the subject an effective amount of a composition comprising a purine analog having inhibitory activity for cdk2 cyclin-dependent kinase, thereby antagonizing short-form c-Maf transcription factor and an acceptable carrier.

15. The method of claim 14 wherein the purine analog comprises purvalanol A, purvalanol B, amino-purvalanol, olomoucine, N9-isopropylolomoucine, roscovitine, methoxy-roscovitine, combinations thereof, or salts thereof.

16. The method of claim 14 wherein the purine analog comprises purvalanol A, purvalanol B, combinations thereof, or salts thereof.

17. The method of claim 14 wherein the purine analog comprises purvalanol A.

18. A method of treatment for steroid-induced glaucoma in a subject, the method comprising administering to the subject an effective amount of a composition comprising a purine analog having inhibitory activity for cdk2 cyclin-dependent kinase, thereby antagonizing short-form c-Maf transcription factor and an acceptable carrier.

19. The method of claim 18 wherein the purine analog comprises purvalanol A, purvalanol B, amino-purvalanol, olomoucine, N9-isopropylolomoucine, roscovitine, methoxy-roscovitine, combinations thereof, or salts thereof.

20. The method of claim 19 wherein the purine analog comprises purvalanol A, purvalanol B, combinations thereof, or salts thereof.
